(19) United States
(12) Patent Application Publication
(10) Pub. No.: US 2025/0032084 A1
(43) Pub. Date: Jan. 30, 2025

(54) PHANTOM WITH RADIOSENSITIVE LESIONS AND PARTIALLY FILLABLE CAVITIES

(71) Applicant: Acoustic Range Estimates, LLC, Chicago, IL (US)

(72) Inventors: Sarah K. Patch, Chicago, IL (US); Kelly A. McElvain, West Allis, WI (US)

(73) Assignee: Acoustic Range Estimates, LLC, Chicago, IL (US)

(21) Appl. No.: 18/780,080

(22) Filed: Jul. 22, 2024

Related U.S. Application Data
(60) Provisional application No. 63/515,585, filed on Jul. 25, 2023.

(51) Int. Cl. A61B 6/58; A61N 5/10

(52) U.S. Cl. CPC A61B 6/583; A61N 5/1075; A61N 2005/1076

(57) ABSTRACT

A phantom for radiotherapy and/or medical imaging systems. The phantom includes one or more simulated anatomical structures including a tissue mimicking soft material. The phantom further includes a radiosensitive inclusion configured to form a bubble when exposed to radiation. The bubble is detectable via imaging with the medical imaging system.

### CROSS REFERENCE TO RELATED APPLICATION

This application claims the benefit of U.S. Provisional Patent Application No. 63/515,585 filed Jul. 25, 2023 is incorporated herein by reference in its entirety.

### FIELD

The present disclosure generally relates to radiotherapy, and more particularly to phantoms used in radiotherapy.

### BACKGROUND

Within the fields of radiotherapy and radiography, specialized objects may be imaged to evaluate, analyze, and/or calibrate a given radiotherapy or imaging device. These specialized objects are often referred to as “phantoms” since they are substitutes for the subject matter ultimately intended to be irradiated or imaged, such as a human patient. It is advantageous to use phantoms because physical properties of the phantom can be quantified by direct measurement. In contrast, physical properties of human tissues vary greatly and can only be inferred in vivo from non-invasive radiographic images. By way of example, phantoms are used to validate dose output of ionizing radiation therapy machines that deliver x-rays, gamma rays, electron beams, and particle beams. Similarly, phantoms are used to validate conventional two-dimensional (2D) planar x-ray images or three-dimensional (3D) images such as single-photon emission computerized tomography (SPECT), magnetic resonance imaging (MRI), computed tomography (CT), ultrasound, positron emission tomography (PET), and other imaging modalities. To date, most phantoms are designed either for radiotherapy or medical imaging. However, radiotherapy treatment planning is based on radiographic images, typically CT. Indeed, the first step in a standard radiotherapy workflow is CT imaging (see FIG. 1). Similarly, phantoms may be used to validate dose output of radiation therapy machines that deliver x-rays, gamma rays, electron beams, and particle beams. U.S. Pat. No. 4,350,607 (Apfel 1979) and U.S. Pat. No. 5,840,276 (Apfel 1998) generally relate to detectors and dosimeters for neutrons and other radiation and activatable infusible dispersions containing drops of superheated liquid for methods of therapy and diagnosis, which are each incorporated by reference herein in entirety.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure generally relates to a phantom for radiotherapy and/or medical imaging systems. The phantom includes one or more simulated anatomical structures comprised of a tissue mimicking soft material. The phantom further includes a radiosensitive inclusion configured to form a bubble when exposed to radiation. The bubble is detectable via imaging with the medical imaging system.

In certain examples, the phantom is configured to be pressurized such that the bubble is re-compressed into liquid form. In further examples, the phantom is configured to withstand at least 100 PSIG for at least 10 minutes. In further examples, the phantom comprises a simulated lung, and wherein the simulated lung is configured to withstand the at least 100 PSIG for the at least 10 minutes.

In certain examples, the one or more simulated anatomical structures comprise a simulated gastrointestinal tract having at least two ports that allow fluid to flow through the phantom. Further examples include plugs operable to seal the at least two ports to selectively seal a liquid and/or a gas therein.

In certain examples, the radiosensitive inclusion is positioned inside at least one of the one or more simulated anatomical structures.

In certain examples, the one or more simulated anatomical structures include at least one simulated lung having high acoustic impedance.

In certain examples, the one or more simulated anatomical structures include a simulated gastrointestinal tract configured to be at least partially fillable.

In certain examples, the radiosensitive inclusions require high linear energy transfer radiation as the radiation to vaporize.

In certain examples, the radiosensitive inclusion comprises a superheated droplet at room temperature.

Certain examples further include a simulated bone having a greater hardness than the tissue mimicking soft material.

Another aspect according to the present disclosure generally relates to a method for making a phantom for radiotherapy and/or medical imaging. The method includes providing a first material with a second material therein, wherein the second material is different than the first material and comprises a tissue mimicking soft material. The method further includes providing a radiosensitive inclusion within at least one of the first material and the second material, wherein the radiosensitive inclusion is configured to form a bubble when exposed to radiation, and wherein the bubble is detectable via imaging with the medical imaging system.

In certain examples, the method further includes providing a simulated gastrointestinal tract through the phantom such that the simulated gastrointestinal tract extends from an entrance to an exit each open at an outside of the phantom, allowing flow through the phantom. Further examples include at least partially filling the simulated gastrointestinal tract with a liquid and/or a gas and closing the entrance and the exit to seal the liquid and/or gas therein.

Certain examples further include, when the radiosensitive inclusion has been vaporized to form the bubble, pressurizing the phantom such that the bubble is re-compressed into liquid form. Further examples include providing a simulated gastrointestinal tract through the phantom such that the simulated gastrointestinal tract extends from an entrance to an exit each open at an outside of the phantom, further comprising unsealing at least one of the entrance and the exit before pressurizing the phantom such that pressure inside the simulated gastrointestinal tract equilibrates with the outside of the phantom. Further examples include pressurizing the phantom comprises applying at least 100 PSIG of pressure for at least 10 minutes.

In certain examples, the bubble is one of multiple bubbles that the radiosensitive inclusion is configured to form when exposed to radiation, and the method further includes calibrating the radiosensitive inclusion so that a number of the multiple bubbles vaporized indicates a radiation dose.

Certain examples further include irradiating the phantom with high linear energy transfer radiation to cause the radiosensitive inclusion to vaporize and form the bubble, and pressurizing the phantom such that the bubble is re-compressed into liquid form.

Certain examples further include providing a simulated lung within the first material, and, when the radiosensitive inclusion has been vaporized to form the bubble, pressurizing the phantom such that the bubble is re-compressed into liquid form, wherein the simulated lung is configured to withstand at least 100 PSIG for at least 10 minutes without damage thereto while the bubble is re-compressed into the liquid form.

Certain examples further include providing a simulated bone within the first material, the simulated bone having a greater hardness than the tissue mimicking soft material.

Another aspect according to the present disclosure generally relates to a phantom for radiotherapy and/or medical imaging systems. The phantom includes a first material and a simulated anatomical structure positioned within the first material, wherein the anatomical structure is comprised of a second material that is different than the first material and comprises a tissue mimicking soft material, wherein the simulated anatomical structure comprises a simulated gastrointestinal tract. The phantom further includes at least two ports in fluid communication with the simulated gastrointestinal tract, wherein the at least two ports allow fluid to flow through the phantom.

Certain examples further include plugs operable to seal the at least two ports to selectively seal a liquid and/or a gas within the simulated gastrointestinal tract.

In certain examples, the one or more simulated anatomical structures further comprise at least one simulated lung having high acoustic impedance.

Certain examples further include a simulated bone within the first material, the simulated bone having a greater hardness than the tissue mimicking soft material. Further examples relate to a method of using the phantom, the method including at least partially filling the simulated gastrointestinal tract with a known volume via at least one of the two ports and imaging the phantom while the simulated gastrointestinal tract is partially filled with the known volume.

It should be recognized that the different aspects described throughout this disclosure may be combined in different manners, including those than expressly disclosed in the provided examples, while still constituting an invention accord to the present disclosure.

Various other features, objects and advantages of the disclosure will be made apparent from the following description taken together with the drawings.

### BRIEF DESCRIPTION OF THE DRAWINGS

FIG. 1 is a flow chart depicting a typical radiotherapy workflow.

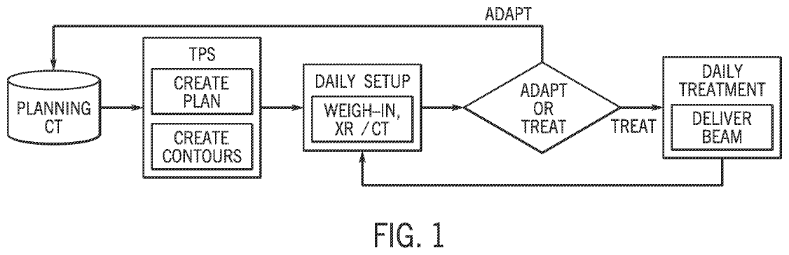

FIG. 2. is a graph depicting relative dose as a function of treatment depth for radiotherapy delivered by x-rays and protons.

FIGS. 3A-3C are graphical representations depicting the sensitivity of particle beam ranges to anatomical changes.

FIG. 4 is a perspective inferior view of a phantom known in the art used for radiotherapy.

FIG. 5 is a perspective inferior view of a phantom according to the present disclosure, shown without valves for filling and emptying the gastrointestinal (GI) system.

FIG. 6 is a perspective inferior view of the phantom of FIG. 5 with a portion of the internal structures removed to show the GI system and valves connected thereto.

FIG. 7 is a sectional anterior view of a phantom such as that shown in FIG. 1.

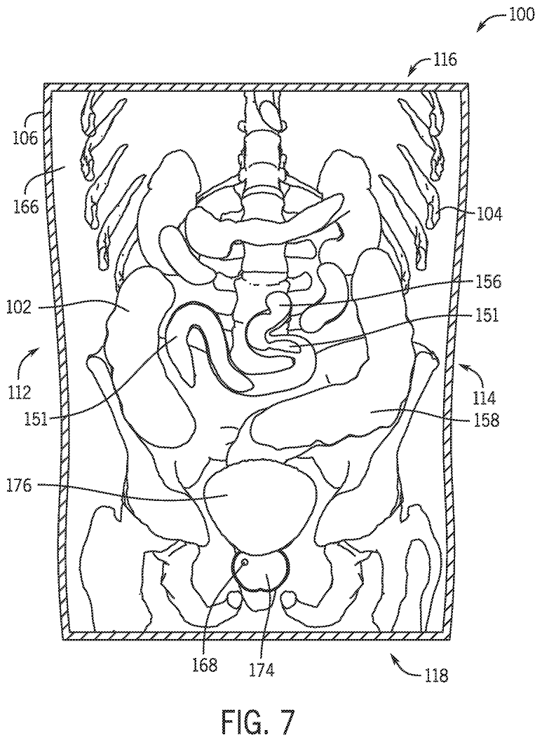

FIG. 8 is a sectional lateral view of a phantom similar to that shown in FIG. 1, in this embodiment not having a shell.

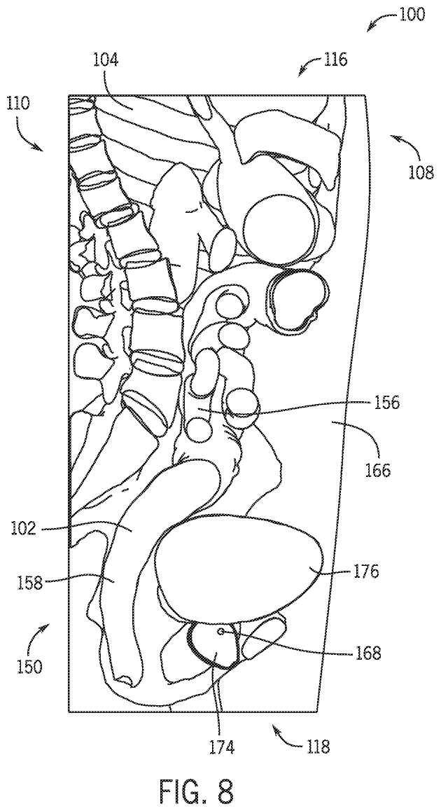

FIG. 9 is a flow chart for one method of using a phantom for a medical imaging system according to the present disclosure.

### DETAILED DESCRIPTION

Oncologists rely on patient imaging to support clinical decision-making and patient care for many types of cancer and other diseases, both for human and non-human subjects or patients. Radiotherapy treatment plans specify energies, incident angles, and intensities of x-ray or charged particle beams that are required to achieve the prescribed dose for a target, while minimizing dose to surrounding healthy tissue. Conventional treatment planning software (TPS) applies a formula to convert the Hounsfield units (HU) value assigned to a CT voxel to quantities describing x-ray attenuation properties or charged particle stopping power. Then, the TPS optimizes the array of deliverable dose maps to best match the prescribed dose, while minimizing collateral damage.

Dose delivered by x-ray beams decreases approximately exponentially, according to Beer's Law. This smooth and nearly exponential decrease in absorbed dose as a function of depth indicates that treatment plans are relatively insensitive to errors in HU-to-attenuation conversion and patient alignment along the beam trajectory.

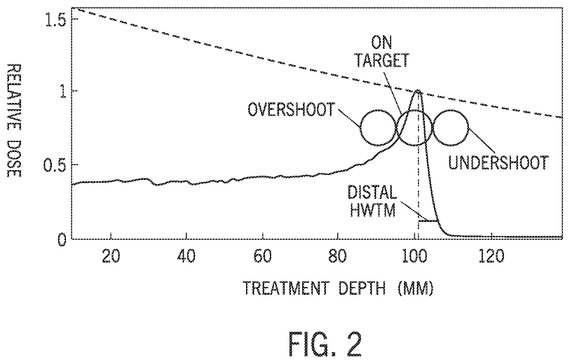

Charged particles lose energy according to the Bethe-Block equation and stop in the patient. Dose delivered varies dramatically along the beam trajectory, dropping within a few millimeters by an order of magnitude from a maximum at the Bragg peak (see FIG. 2, whereby the dashed line represents the relative dose from photons while the bold line represents that from protons). Although collateral damage caused by particle therapy plans is lower than that of x-ray treatment plans, misalignment risk is greater. Furthermore, the HU-to-stopping power conversion is not governed by a constant, consistent, or even slowly varying function. Rather, materials that generate the same HU value in a CT scan can have significantly different stopping power.

To ensure tumor coverage despite treatment planning errors, range margins are added around the target. Different clinics apply different margins, computed as a percentage of the beam range. Typical range margins may be up to 3.5% of the beam range in the patient. To reduce range margins, new imaging techniques like dual-energy CT (DECT) and multi-spectral CT are significant topics of research. Additionally, sharp dose falloff of the Bragg curve implies that changes in patient anatomy pose a great risk to proton therapy patients. Day-to-day changes are often referred to as “interfraction” changes; sudden changes that occur during a treatment session are referred to as “intrafraction” changes.

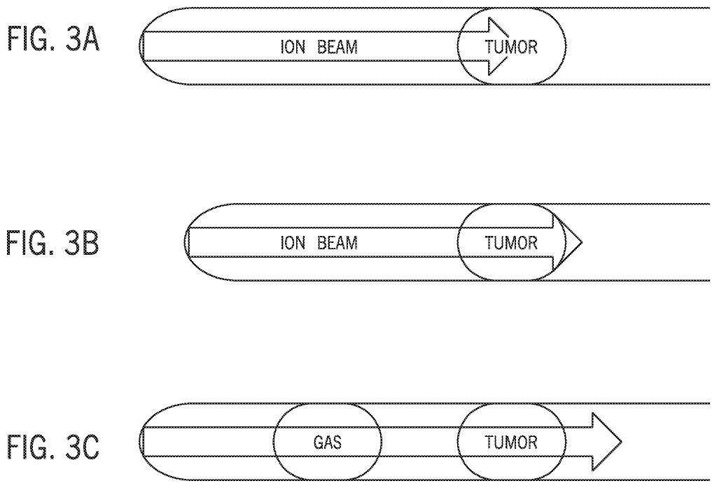

The most common causes of inter-fraction anatomical change are weight loss and digestion. FIG. 3A depicts a particle beam stopping in the center of a tumor as planned and intended. However, cancer patients often lose weight during the weeks-long course of therapy, reducing the fat layer and beam range required to hit a tumor, resulting in overshoot as shown in FIG. 3B. Similarly, bowel gas can increase beam range in the patient, also causing overshoot as shown in FIG. 3C (which may be additive with the overshoot of FIG. 3B). Therefore, a standard procedure is to weigh patients each day before therapy and perform in-room imaging (typically planar x-ray or CT) before each patient session to monitor for weight loss and bowel gas.

Common causes of intra-fraction anatomical change are respiratory and cardiac motion, both of which can be managed by gating techniques. Sudden movement of bowel gas can also cause intra-fraction anatomical change. To ensure complete tumor coverage absolute range margins of 1-3 mm (for example) are often added to guard against anatomical changes and also errors in patient setup. To reduce these margins, in-room imaging and range verification devices are under development.

To reduce both percentage range and absolute setup margins, the present inventors recognize that new range verification techniques are required. Proper calibration of imaging, radiotherapy, and range verification devices is crucial. Inter-comparison of systems located in different institutions is crucial for clinical research rigor. As such, quantitative radiotherapy phantoms are needed to validate those techniques.

Traditional phantoms are manufactured from human-made materials such as acrylic, resins, hydrogels, and others. By way of example, solid water (SunNuclear), virtual water (MedCal/Standard Imaging), are rigid non-anthropomorphic phantoms, 3D printing resin such as VeroClear™ (Stratasys® Ltd, Eden prairie, MN, USA), and thermoplastic polymer such as polyglycolic acid (PGA), respectively, have been used in phantom production in radiology applications.

Traditional phantoms are often modality-specific. For example, gelatin phantoms are typically produced for ultrasound imaging, and liquid phantoms are frequently used for MRI. In contrast, radiotherapy and x-ray CT phantoms are typically made of resins or hard materials, such as Delrin (or in certain cases, water-filled phantoms). For various reasons, including their bulk, phantoms are typically not concurrently treated or imaged with the patient.

High-quality radiotherapy phantoms are produced with carefully controlled material composition from which quantitative dosimetry data can be both computed and measured. Dosimetry data can include x-ray attenuation and stopping power of charged particles over a range of energies. In terms of x-ray attenuation and/or proton stopping power, these phantoms contain tissue-equivalent materials and also materials used in implants, e.g. stents, pacemakers, orthopedic screws, and joints.

The present inventors recognized problems with phantoms presently known in the art. As soft tissue imaging (MRI, ultrasound) is integrated into radiotherapy practice (Elekta's Clarity, Elekta Unity, and MRIdian), radiotherapy phantoms will need multi-modality capability. Not only should they mimic x-ray attenuation of biological tissue, but also ultrasound and MRI tissue properties. In particular, the present inventors identified that phantoms known in the art provide very poor representations of some soft tissues and pathways within the body, including for example the GI system (also referred to as simply the intestines). This is due in part to the nature of the anatomy comprising soft tissue and following a long and tortuous path in all three dimensions. This is further exacerbated because the intestines of real patients include a mixture of gases, liquids, and solids. Additionally, this mixture is not static, but changes over time.

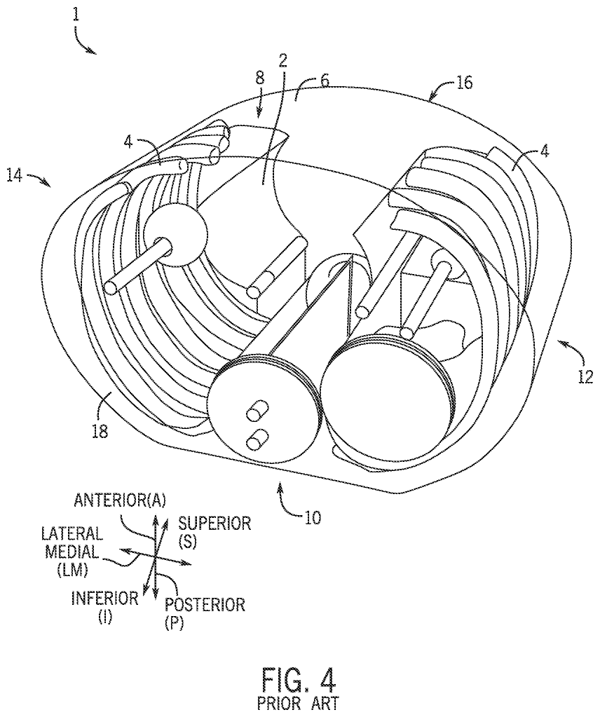

One anthropomorphic phantom well known in the art is the Sun Nuclear 057a, which was designed only for multi-modality imaging (CT, MRI, ultrasound) and has been used in research for developing range verification devices. The Sun Nuclear 057a model is solid and does not contain intestines. FIG. 4 depicts an example of another phantom 1 known in the art, specifically model E2E SBRT produced by Sun Nuclear, part of Mirion Medical Company of Melbourne, FL, USA. The phantom 1 represents a simplified anatomy of a small adult, and is made of proprietary epoxy resins that are hard when cured. The phantom includes ribs 4 contained within a housing 6. The housing 6 has an anterior side 8 and a posterior side 10, left side 12 and right side 14, and a superior side 16 and an inferior side 18 representing a portion of a patient's torso.

New proton therapy installations are increasingly common, due primarily to the 2016 introduction of single-room proton therapy systems with synchrocyclotrons, which are an order of magnitude less costly than multi-room systems with conventional accelerators. Accordingly, the present inventors recognize that the need for particle therapy phantoms is not only present but is growing at a rapid rate along with the rise in new installations.

As such, the present inventors have recognized an unmet need for phantoms having the following properties: 1) accurately simulates the gastrointestinal tract and can be filled as needed with liquids, solids, and/or gases; 2) includes radiosensitive inclusions (also referred to as droplets) that can be reused over time; 3) has known x-ray attenuation and ion stopping power for treatment planning.

Potential utility of quantitative radiotherapy phantoms has increased due to improved Monte Carlo modeling capabilities of treatment planning systems. For instance, RayStation® (RaySearch Laboratories, Stockholm, Sweden) now enables accurate treatment plans for phantoms with known physical properties: chemical composition (stoichiometry), mass density, and mean excitation energy.

The present inventors have also identified that the introduction and improvement in additive manufacturing (AM) and 3D printing technology that now enable new improvements in efficient design and repeatable manufacturing of complicated geometric objects. For instance, these new technologies could be utilized to produce anatomically correct ribs, made of rigid material, that curve around vital organs. 3D printing of soft tissues, like the intestines is more challenging, but PolyJet™ technology (Stratasys Ltd, Eden Praire, MN, USA) shows great promise. Flexible resins and multiple materials can be utilized in a single print. PolyJet printers ensure increased attention to detail for complex geometry applications where fine resolution and smooth surface finish prints are desirable. This could be useful in accurately mimicking anatomic texture and geometries in anthropomorphic phantom fabrication.

In view of this, the present inventors have developed the presently disclosed phantoms, methods of manufacturing such phantoms, and methods of their use, which not only improve the deficiencies described above, but are also contemplated to 1) enable validation of range verification devices developed for particle therapy; 2) serve as training phantoms with which clinicians can practice before treating a patient. In particular, an anthropomorphic phantom with fillable GI tract may be useful for testing range verification device performance subject to a sudden (intra-fraction) change in bowel gas. Similarly, the phantom may be used to test adaptive and robust planning protocols against interfraction changes in bowel gas. It should be recognized that the present disclosure is not limited to phantoms, methods of manufacturing, and methods of use that include all of the properties listed above, nor provide all of the contemplated benefits listed above.

Certain examples of phantoms disclosed herein provide for tissue mimicking gelatin phantoms in which the simulated intestines can mimic filling and presence of bowel gas. Additionally, certain examples of phantoms described herein provide two unique features that provide utility for radiation therapy, especially particle therapy.

By way of example for a phantom that includes hard and soft materials, Table 1 below provides material properties for different materials used in the Sun Nuclear 057a. that are suitable for creating portions of a phantom according to the present disclosure. Generally, organs, background material, fat and muscle can be made of hydrogels similar to those used in existing phantoms such as the Sun Nuclear 057a (shown in Table 1 below). Only the bone is made of a hard material.

TABLE 1
Properties of Example Materials for Organs, Background Material,
and Bone in the Sun Nuclear 057a

| | | νₛ | ρ | C | β | |
|---|---|---|---|---|---|---|
| | HU range | (m/s) | (kg/m³) | (MJ/m\hat{ }3/K) | (1/K) | Γ |
| Fat | (−200, −25) | 1430 | 894 ± 2.64 | 1.61 ± 0.07 | 1.0e−3 | 1.14 |
| Background | (−25, 46) | 1540 | 1040 ± 0.10 | 3.83 ± 0.44 | 3.5e−4 | 0.23 |
| lesion-standard | (−25, 46) | 1540 | 1025 ± 0.19 | 4.69 ± 0.73 | 3.5e−4 | 0.18 |
| Muscle | (46, 68) | 1555 | 1053 ± 9.66 | 3.89 ± 0.29 | 3.5e−4 | 0.23 |
| Liver/kidney/vessel | (68, 100) | 1540 | 1084 ± 0.34 | 4.91 ± 0.96 | 3.5e−4 | 0.18 |
| Vein | (100, 200) | 1560 | 1053 ± 0.70 | 2.99 ± 0.25 | 3.5e−4 | 0.29 |
| Bone | (200, inf) | 3200 | 1313 ± 6.11 | 1.65 ± 0.04 | 4.0−4 | 3.28 |
| Water/Aquasonic | NA | 1480 | 1000 | NA | | NA |

During experimental testing of a range verification prototype using the Sun Nuclear 057a, the HU-to-proton stopping power curve for patients provided positive preliminary data. Thermoacoustic emissions generated during delivery of that plan were simulated using material properties in Table 1 agreed well with simulated emissions and provided confidence that hydrogels inside the phantom attenuated x-rays similarly to human tissue. In certain embodiments, the present inventors have recognized that the accuracy of treatment plans developed for phantoms may be further improved by defining stopping power in each voxel based on elemental composition and ionization energy of the material in that voxel. This is practiced routinely by basic scientists using Monte Carlo (MC) simulation software such as TRIM and GEANT, and has been adopted by (at least) one TPS (RayStation).

Certain phantoms according to the present disclosure include a tortured cavity that runs through the phantom, mimicking a gastrointestinal tract. The cavity has entry and exit points through which fluid can flow, e.g. via a peristaltic pump such as those used during proton treatments. The cavity advantageously allows clinicians and researchers to mimic partial filling of the intestines, the variation of which can induce large range errors during particle therapy if not quantified and corrected. As described further below, the disclosed phantoms can tolerate high hydrostatic pressure when the GI tract is filled with fluid. The present inventors have recognized that equalizing pressure inside the GI tract ensures that the GI cavity does not collapse when pressure is applied to re-compress vaporized droplets.

Certain phantoms according to the present disclosure also or alternatively include a radiosensitive gel infused with superheated droplets. By way of example, the radiosensitive gel may be that used in Bubbletech Personal Neutron Dosimeter (PND) detectors (Bubble Technology Industries (BTI) of Ontario, Canada). These detectors were designed to be worn in a shirt pocket. At the end of each workday, the employee “counts” the number of bubbles visible to the naked eye and resets the detector by screwing the top cap to apply pressure to liquify any vaporized gas. When starting a new workday the cap is unscrewed, reducing pressure so that the room temperature device is now above critical temperature of the liquefied nanodroplets. When exposed to neutrons, droplets in the PND detectors vaporize.

When such “droplet detectors” are exposed to high linear energy transfer (LET) radiation, a small fraction of the unstable superheated droplets are vaporized. Recently, it has been shown that x-rays are capable of activating certain compressed nanodroplets (Falatah et al. 2022). The number of visible bubbles increases with dose delivered to the phantom. Such radiosensitive gels can advantageously be re-used, specifically by compressing the bubbles back into droplets.

The present inventors have recognized that there are advantages to quantifying the extent of bubble production via ultrasound imaging rather than using optical readout. Unlike BTI's small and optically clear detectors, anthropomorphic phantoms are large and typically visibly opaque. Accordingly, optically counting the number of bubbles produced is impractical. At minimum, an anthropomorphic radiotherapy phantom must generate tissue mimicking Hounsfield units when imaged by CT. Adding tissue mimicking contrast for ultrasound and or MRI imaging further enhances the utility of a radiotherapy phantom. Therefore, the inventors designed phantoms for which diagnostic imaging techniques like CT, ultrasound, or MRI can be used to assess the number of vaporized nanodroplets.

Phantom and experiment design should account for the imaging technique used to count vaporized bubbles. Vaporized bubbles are strong acoustic reflectors. Therefore, small bubbles can serve as point spread functions of ultrasound arrays, because resulting ultrasound images represent the system's attempt to image a delta-function. Small bubbles appear in images as bright spots with diameter determined by the resolution of the ultrasound array rather than the bubble. Therefore, the radiosensitive lesions and radiotherapy measurements should be designed so that the odds of two vaporized bubbles being separated by less than the resolution of the ultrasound is small. The fraction of nanodroplets that vaporizes increases with dose delivered. Experiment design should ensure sufficient dose is delivered to vaporize enough droplets to ensure adequate bubble counts, while minimizing the incidence of duplicate bubbles within the resolution of the ultrasound array. High-resolution x-ray CT can detect larger bubbles, with O(1 mm) diameter, but may fail to detect bubbles with O(100 micron) diameter. Therefore, phantoms designed for experiments using only CT should ensure that vaporized nanodroplets grow to at least O(1 mm) diameter.

Table 2 contains compounds that are superheated at room temperature and atmospheric pressure when liquefied. The first compounds, Freon 114 and Freon 12, have been frequently used. However, they are environmentally unfriendly and are no longer approved for widespread use.

TABLE 2
Superheated compounds at room temperature

| | boiling | | |
|---|---|---|---|
| | point | vapor pressure & | |
| compound | (° C.) | temperature | comments |
| C2Cl2F4“Freon 114” | +3.8 | 1.9 atm @ 21° C. | no longer mass produced |
| CCl2F2 “Freon 12” | −29.8 | 5.6 atm @ 20° C. | no longer mass produced |
| C3H2F4 “tetrafluoropropene” | −30 | 6 atm @ 21° C. | |
| C4F10 “perfluorobutane” | −1.7 | 3.3 atm @ 25° C. | gas in Sonazoid & Optison |
| | | | microbubbles |
| C3F8 “octafluoropropane” | −37 | 7.9 atm @ 21° C. | gas in Definity microbubbles |

Examples of media into which nanodroplets are dispersed were disclosed in the patents incorporated by reference above. More recently, nanodroplets have been dispersed in hydrogel phantoms thickened using Carbopol. By way of example, media that may be used within the phantom 100 include tissue mimicking soft material, such as the gelatins developed by Madsen, Sun Nuclear (e.g., within its Sono Ultrasound Phantoms) or Zerdine®. Aquasonic 100 Ultrasound Transmission Gel or ballistic gels can be used to mimic anechoic tissue like a full bladder 176.

The present inventors and others have investigated the possibility of using microbubble contrast agents, such as Sonazoid™ by General Electric or Definity™ by Lantheus to improve radiation therapy.

Certain embodiments of phantoms according to the present disclosure incorporate superheated nanodroplets to add dosimetric capability while maintaining tissue mimicking properties. It has been shown that stopping ions vaporized superheated nanodroplets of gasses used in microbubble contrast agents. At body temperature, secondary nuclear emissions generated by stopping protons re-vaporize liquified microbubbles, i.e., nanodroplets of perfluorobutane, proximal to the Bragg peak. Stopping carbon ions are capable of directly vaporizing the same nanodroplets at the Bragg peak.

The octafluoropropane used in Definity would have greater superheat at body temperature than the perfluorobutane used in Sonazoid and Optison. Therefore, the present inventors determined that the droplets liquefied from the Sonazoid microbubbles are expected to vaporize proximal to the Bragg peak. Droplets liquefied from Definity have a higher degree of superheat and are more likely to vaporize in the Bragg peak. The present inventors have further recognized that the particular nanodroplets of gases may be selected to be optimal for creating phantoms.

In certain embodiments, calibration may be performed by comparing data provided from dosimeters (TLDs, diode arrays, ionization chambers) to bubble counts in small samples of nanodroplet-infused gel. By way of example, the Delta4 Phantom+(ScandiDos, Inc., Ashland, VA, USA), comprised of two orthogonal silicon diode arrays, is utilized to measure dose distribution for several quality assurance (QA) applications. The IC-Profiler™ (Sun Nuclear Corporation, Melbourne, FL, USA) is an array of ion chambers for linear accelerator (linac) QA. Rather than delivering a high-energy proton beam through stacked dosimeters of the same type, (IBA Zebra stacked ionization chambers), beam could be delivered through a stack of interleaved dosimeters and radiosensitive gel. Because the Bragg curve for high energy protons is smooth, the concentration of bubbles nucleated as a function of radiation dose, LET, and fluorocarbon concentration can be determined by interpolating between dosimeter readings. The number of bubbles in each gel sample may be counted by high-resolution ultrasound imaging. Echogenicity of ultrasound images may be compared to optical counts in sufficiently small transparent samples. By way of example, optical counts could be obtained via visual inspection and/or the use of a light microscope.

The present inventors have hypothesized that if the phase change of perfluorobutane, or other superheatable gases used in contrast agents, is as reversible as that of commercial detectors, then applying high pressure to microbubble contrast agents will liquefy the gas cores into nanodroplets, which can then be dispersed into a background gel. Additionally, the present inventors have determined that liquefying Sonazoid microbubbles would likely not require applying as much pressure as Definity because Definity has a lower boiling point.

The present inventors recognized that to be cost effective, the phantoms must be designed for multiple uses. For phantoms containing superheated droplets, re-use would necessitate applying high pressure to re-liquify vaporized droplets. However, the present inventors have identified that anatomical phantoms known in the art, such as the Sun Nuclear 057a model phantom, cannot withstand compression. The lung-mimicking material therein is low-density and highly compressible and would be crushed by 100 PSIG.

One solution enabling re-use identified by the present inventors is to produce the lungs from an incompressible material. The present inventors have identified that acoustic impedance, Z=ρνₛ, is inversely related to compressibility κ=1/ρνₛ²=1/Zνₛ. Materials with acoustic impedance that is either higher (bone, metals) or lower (lung, bowel gas) than that of soft tissue induces acoustic reflections. Additionally, the present inventors have identified that replacing low impedance lung mimicking material with high impedance material reverses polarity of acoustic reflections. Therefore, in certain embodiments, low density lung material is replaced by incompressible material with high acoustic impedance, such as a metal (e.g., aluminum, copper, brass, etc.). By way of example, the acoustic impedance of most soft tissues is below 2 MRayls, bone is (nominally) around 8 MRayls, and metals exceed 10 MRayls. Therefore, for the present disclosure the term “high” acoustic impedance may be interpreted as exceeding 4, 6, 7, or 8 MRayls. Typically, high acoustic impedance materials also have strong x-ray stopping power, and thus make CT slices through the lungs very noisy. However, neither of these effects impact thermoacoustic range estimates. In particular, proton treatment plans depend only upon HU values within the beam trajectory, and very few plans for abdominal organs involve paths through the lungs. Furthermore, time shifts between measured and simulated emissions rely upon early arrivals, so reflected signals are not considered.

Similarly, fully enclosed phantoms with partially filled GI tract would not withstand pressurization. The materials and design selected for phantoms according to the present disclosure were chosen to accommodate high pressure without crushing the GI tract. Although pressurization could be achieved multiple ways, a hydrostatic pressure chamber is assumed in this example. Both superior and inferior ends of the GI tract are fully opened before placing the phantom into a hydrostatic pressure chamber. This provides equilibrium both inside and outside the GI tract. The pressure in the hydrostatic chamber is slowly increased, thereby increasing the pressure in the GI tract and throughout the phantom. The equalized pressure inside and outside the GI tract preserves the morphology of the GI tract while vapor bubbles within the phantom are recompressed into liquid droplets. The reversibility of vaporization of superheated droplets increases opportunities for repeated dose delivery, extending reusability of the abdominal phantom. It should be recognized that pressure required to re-liquify vaporized nanodroplets depends upon ambient pressure and temperature, as well as the type of gas being compressed into a nanodroplet. In the example of Bubbletech PNDs at room temperature and atmospheric pressure, applying an additional 200 PSIG for 30 minutes returns vaporized bubbles to their liquid state pre-irradiation. This applied pressure and duration is merely an example and others are contemplated by the present disclosure, including at least 100 PSIG for at least 10 minutes, by way of example.

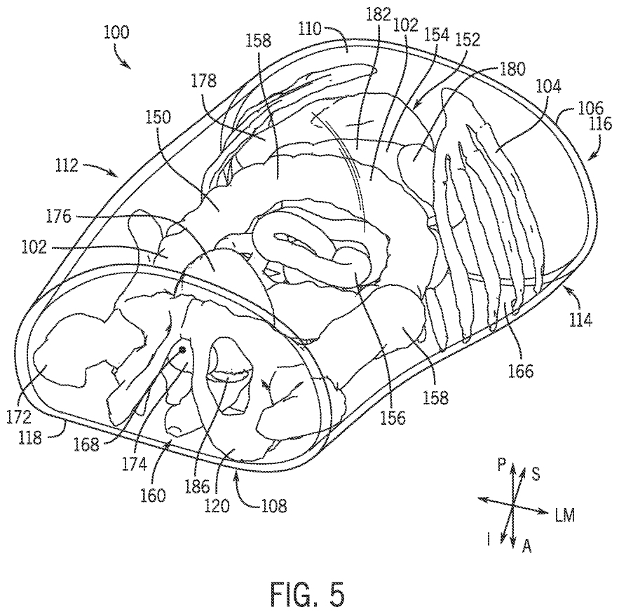

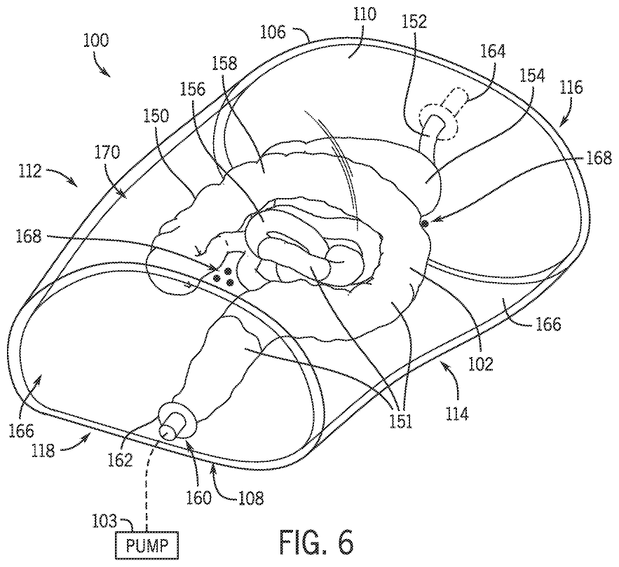

Through experimentation and development, the present inventors produced functional and anthropomorphic phantoms for thermoacoustic range verification (Patch et al. 2016). A more sophisticated example is the phantom 100 of FIGS. 5 and 6. This anthropomorphic phantom 100 was designed to provide a complete material characterization for proton therapy, to incorporate multiple-use dosimetric capability, and to quantify the impact of bowel gas.

The inventors designed phantom 100 to have a more enhanced morphology than other phantoms known in the art, such as the Sun Nuclear 057a, which suffer from anatomically incorrect parallel ribs. In contrast, the new phantom 100 of FIGS. 2-3 includes a more realistic rib cage 104.

A pancreas was also added as the present inventors recognized that outcomes for pancreas cancer patients who received dose-escalated x-ray radiation therapy have been good. However, dose escalated x-ray radiation delivers significant dose to healthy abdominal organs, and particle therapy can reduce that collateral damage. Additionally, the phantom 100 is longer in the SI directions than those presently known in the art, e.g., the Sun Nuclear 057a phantom, to enable oblique ultrasound imaging that keeps acoustic hardware out of the beam during delivery to axial planes.

The phantom 100 once again represents a simplified anatomy of a small adult, which has background material 166 with other materials positioned therein. The background material may be selected to mimic fat and/or skin, which by way of example may be the same used as background material for phantoms known in the art, such as the Sun Nuclear 057a phantom. The second material positioned within the background material 166 may tissue mimicking soft materials 102, such as hydrogels (e.g., Zerdine® Hydrogel produced by Sun Nuclear, part of Mirion Medical Company of Melbourne, FL), QA phantoms such as the SunNuclear GS54, or tissue mimicking gelatins like those developed by Madsen et al. (Madsen, Zagzebski, and Frank 1982; D'Souza et al. 2001; Lazebnik et al. 2005). These soft materials 102 may be selected to mimic different organs (e.g., parts of a gastrointestinal tract, a liver, etc.), as discussed further below.

Like the human torso it is designed to mimic, the soft tissues of the phantom 100 are flexible and susceptible to damage. Acute injury can be caused during transport by puncture or abrasion. Long-term, chronic, injury like sagging due to gravity and/or desiccation. Therefore, in certain examples the phantom is at least partially enclosed with a shell 106The shell 106 may be rugged, stiff material to serve as an exoskeleton, for example being made of plastic. Acoustic imaging and range verification techniques require acoustic coupling to the phantom. The housing could be designed with a few small acoustic windows, but that would limit utility of the phantom to those pre-defined acoustic windows. Alternatively, the housing may be comprised of interlocking or overlapping sections that may be removed as needed to provide acoustic access to a small surface, while maintaining protection and support for most of the phantom's exterior surface. It is anticipated that the posterior, inferior and superior surfaces of the shell 106 might be permanent, but the anterior, left and right would be modular and removable in small segments. In certain embodiments, watertight seals (o-ring or perhaps silicone barriers) between segments will minimize desiccation and maximize phantom shelf life. The present disclosure contemplates embodiment of phantoms that differ from those expressly shown in the drawings, including adding or omitting shells corresponding thereto.

The phantom 100 has an anterior side 110 and a posterior side 108, right side 112 and left side 114, and a superior side 116 and an inferior side 118 representing a portion of a patient's torso. Within the first material 166 of the phantom 100 are different anatomical structures, such as pelvis 120, a liver, two kidneys, lower lung, and spine. In certain embodiments, the phantom also includes blood vessels (not shown). The phantom 100 shown includes a GI system or GI tract 150 that extends from an inferior portion of the esophagus 152, the stomach 154, small intestines 156, large intestines 158, and to a rectum 160. Valves 162, 164 (also referred to as ports) are provided at the two ends of the GI tract 150, which can be connected to a source of fluid (like water) outside the phantom 100. By way of example, the ports may be John Guest or other commercially available types of fluid connections, which may also include features for allowing, blocking, or metering flow rates therethrough. This allows the valves 162, 164 to be used for filling and emptying the GI tract 150 with semi-solids, liquids, and gases as desired (collectively shown as contents 151). One or both valves 162, 164 may be coupled to a pump 103 to fill or empty the GI tract. Both valves should be open during recompression to ensure pressure equalization.

The phantom 100 contains organ, muscle, fat, and bone-mimicking materials as well as space-filling background media 166 between them. By way of example, these additional anatomical structures include part of a femur 172, prostate 174, bladder 176, lung 178, spleen 180, stomach 182, large intestine 184, and seminal vesicles 186. Bulk soft tissues (organs, muscle, and fat) may be made from hydrogels such as Zerdine® (Sun Nuclear) or tissue mimicking gelatin (Madsen, Zagzebski, and Frank 1982; Lazebnik et al. 2005; D'Souza et al. 2001). Six anatomically correct ribs 104 are also provided, which may be formed via 3D printing or other techniques known in the art (as with the other structures, including the background material, shell, and different soft tissues). By way of examples, Delrin rods have been used to mimic straight bone in phantoms designed for x-ray CT and anatomically correct ribs could be found in the pediatric phantom.

Superheated nanoparticles 168 may be suspended throughout soft tissues in the phantom. Alternatively, they may be suspended only in specific organs of interest, or more specifically within tumor-mimicking lesions within individual organs.

During experiments using Bubbletech PNDs, the present inventors identified that the radiosensitive droplets may be hypoechoic compared to background gelatin in high-frequency (7-11 MHz) ultrasound images. Nanodroplets could not be discerned from the background gel in low-frequency (1-4 MHz) ultrasound images, or in x-ray CT images. CT HU values of the PND background material were closer to HU values of metal, rather than human tissue. Standard metal artifact reduction (MAR) reconstruction as known in the art eliminated most streak artifacts from CT images of a phantom in which Bubbletech PND gel was embedded as a radiosensitive lesion.

The present disclosure contemplates several uses for the phantoms disclosed herein. For instance, it may be used for testing the impact of bowel gas during particle therapy. By controlling the filling/emptying of the GI tract 150 via small peristaltic pumps 103 controlled remotely, accuracy of range estimation techniques can be quantified. Range estimates may be obtained before, during, and after filling with known quantities of fluid. For example, an experiment may be designed so that fluid is drawn from a clear graduated cylinder. A time-stamped video may be used to record the volume in the graduated cylinder was compared to time-stamped log files to determine the volume of water in the phantom when each spot is being treated. CT images may be obtained with the same quantities of fluid in the GI tract 150. The accuracy of each thermoacoustic range estimate may be computed from MC simulations corresponding to the volume of fluid in the phantom 100 at the time the spot is treated.

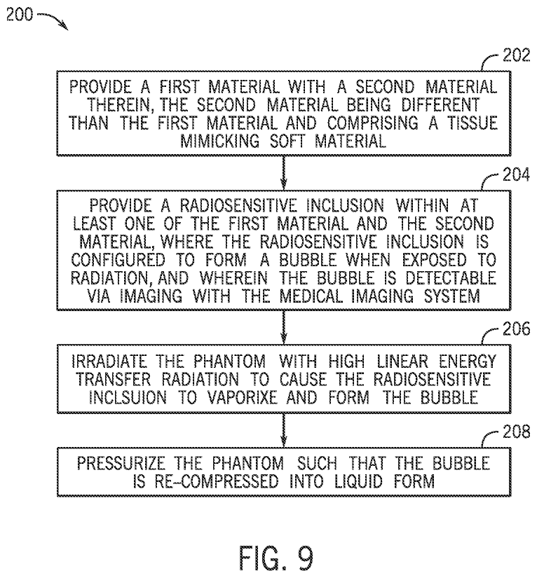

An example of a phantom production method 200 according to the present disclosure is shown in FIG. 9. Step 202 recites providing a first material with a second material therein, whereby the first and second materials are different than each other. By way of example, the first material may be selected to mimic skin or fat, whereas the second material may be selected to mimic an anatomic structure such as an organ (e.g., a GI tract or a liver). The first material may be the outermost portion of the phantom. In other examples, the first material (e.g., which may be a background media 166 such as hydrogel) may be positioned within an optional shell formed of yet another material). This shell may be rigid and may fully enclose the first material therein. In other examples including an optional shell, the shell may not completely enclose the first material, but rather be present at only some of the phantom's ends. Step 204 recites providing a radiosensitive inclusion (e.g., infused with superheated nanoparticles 168 described above) within the first material, wherein the radiosensitive inclusion is configured to vaporize to form bubbles when exposed to radiation, and wherein the bubbles are detectable via imaging with the medical imaging system. Step 206 provides for irradiating the phantom with high LET radiation to cause the radiosensitive inclusion to vaporize and form the bubble, followed by step 208 of pressurizing the phantom such that the bubbles are re-compressed into liquid form as in a manner described above. It should be recognized that this process would not be compatible with phantoms known in the art. For example, for phantoms that include simulated lungs, these lungs are made of lung-mimicking material that is low-density and highly compressible, which would be crushed by 100 PSIG as stated above.

Although the patient-facing practice of radiotherapy is not directly impacted by phantoms, indirect benefits to patients can be substantial. Range verification devices can be tested during development. Repeatability and reproducibility of finalized range verification devices can be assessed using phantom measurements. Additionally, robust planning protocols can be tested using a phantom with radiosensitive lesions and partially fillable GI tract. Institution-specific procedures and equipment can be tested and validated using physical phantoms. Finally, anthropomorphic phantoms are used for training purposes in many fields, including radiotherapy.

For the sake of brevity, certain terms have been used throughout the present disclosure that may also be described with other terms. By way of non-limiting example, these include the following: scan and image; day-to-day, interfraction, and chronic; daily and intrafraction; GI tract, intestine, colon, bowel; charged particle, ion, proton, and carbon ion; stopping protons, breaking protons, stopping ions, and breaking ions; droplet, nanodroplet, and liquified microbubble; tumor, inclusion, lesion, and solid mass; port, entrance, entry and exit; and point spread function and impulse response function.

This written description uses examples to disclose the invention, including the best mode, and also to enable any person skilled in the art to make and use the invention. Certain terms have been used for brevity, clarity, and understanding. No unnecessary limitations are to be inferred therefrom beyond the requirement of the prior art because such terms are used for descriptive purposes only and are intended to be broadly construed. The patentable scope of the invention is defined by the claims and may include other examples that occur to those skilled in the art. Such other examples are intended to be within the scope of the claims if they have features or structural elements that do not differ from the literal language of the claims, or if they include equivalent features or structural elements with insubstantial differences from the literal languages of the claims.

What is claimed is:

1. A phantom for radiotherapy and/or medical imaging systems, the phantom comprising:
one or more simulated anatomical structures comprised of a tissue mimicking soft material; and
a radiosensitive inclusion, wherein the radiosensitive inclusion is configured to form a bubble when exposed to radiation, wherein the bubble is detectable via imaging with the medical imaging system.

2. The phantom according to claim 1, wherein the phantom is configured to be pressurized such that the bubble is re-compressed into liquid form.

3. The phantom according to claim 2, wherein the phantom is configured to withstand at least 100 PSIG for at least 10 minutes.

4. The phantom according to claim 3, wherein the phantom comprises a simulated lung, and wherein the simulated lung is configured to withstand the at least 100 PSIG for the at least 10 minutes.

5. The phantom according to claim 1, wherein the one or more simulated anatomical structures comprise a simulated gastrointestinal tract having at least two ports that allow fluid to flow through the phantom.

6. The phantom according to claim 5, further comprising plugs operable to seal the at least two ports to selectively seal a liquid and/or a gas therein.

7. The phantom according to claim 1, wherein the radiosensitive inclusion is positioned inside at least one of the one or more simulated anatomical structures.

8. The phantom according to claim 1, wherein the one or more simulated anatomical structures include at least one simulated lung having high acoustic impedance.

9. The phantom according to claim 1, wherein the one or more simulated anatomical structures include a simulated gastrointestinal tract configured to be at least partially fillable.

10. The phantom according to claim 1, wherein the radiosensitive inclusions require high linear energy transfer radiation as the radiation to vaporize.

11. The phantom according to claim 1, wherein the radiosensitive inclusion comprises a superheated droplet at room temperature.

12. The phantom according to claim 1, further comprising a simulated bone having a greater hardness than the tissue mimicking soft material.

13. A method for making a phantom for radiotherapy and/or medical imaging, the method comprising:
providing a first material with a second material therein, wherein the second material is different than the first material and comprises a tissue mimicking soft material; and
providing a radiosensitive inclusion within at least one of the first material and the second material, wherein the radiosensitive inclusion is configured to form a bubble when exposed to radiation, and wherein the bubble is detectable via imaging with the medical imaging system.

14. The method according to claim 13, further comprising providing a simulated gastrointestinal tract through the phantom such that the simulated gastrointestinal tract extends from an entrance to an exit each open at an outside of the phantom, allowing flow through the phantom.

15. The method according to claim 14, further comprising at least partially filling the simulated gastrointestinal tract with a liquid and/or a gas and closing the entrance and the exit to seal the liquid and/or gas therein.

16. The method according to claim 13, further comprising, when the radiosensitive inclusion has been vaporized to form the bubble, pressurizing the phantom such that the bubble is re-compressed into liquid form.

17. The method according to claim 16, further comprising providing a simulated gastrointestinal tract through the phantom such that the simulated gastrointestinal tract extends from an entrance to an exit each open at an outside of the phantom, further comprising unsealing at least one of the entrance and the exit before pressurizing the phantom such that pressure inside the simulated gastrointestinal tract equilibrates with the outside of the phantom.

18. The method according to claim 17, wherein pressurizing the phantom comprises applying at least 100 PSIG of pressure for at least 10 minutes.

19. The method according to claim 13, wherein the bubble is one of multiple bubbles that the radiosensitive inclusion is configured to form when exposed to radiation, further comprising calibrating the radiosensitive inclusion so that a number of the multiple bubbles vaporized indicates a radiation dose.

20. The method according to claim 13, further comprising irradiating the phantom with high linear energy transfer radiation to cause the radiosensitive inclusion to vaporize and form the bubble; and
pressurizing the phantom such that the bubble is re-compressed into liquid form.

21. The method according to claim 13, further comprising providing a simulated lung within the first material, and further comprising, when the radiosensitive inclusion has been vaporized to form the bubble, pressurizing the phantom such that the bubble is re-compressed into liquid form, wherein the simulated lung is configured to withstand at least 100 PSIG for at least 10 minutes without damage thereto while the bubble is re-compressed into the liquid form.

22. The method according to claim 13, further comprising providing a simulated bone within the first material, the simulated bone having a greater hardness than the tissue mimicking soft material.

23. A phantom for radiotherapy and/or medical imaging systems, the phantom comprising:
a first material;
a simulated anatomical structure positioned within the first material, wherein the anatomical structure is comprised of a second material that is different than the first material and comprises a tissue mimicking soft material, wherein the simulated anatomical structure comprises a simulated gastrointestinal tract; and
at least two ports in fluid communication with the simulated gastrointestinal tract, wherein the at least two ports allow fluid to flow through the phantom.

24. The phantom according to claim 23, further comprising plugs operable to seal the at least two ports to selectively seal a liquid and/or a gas within the simulated gastrointestinal tract.

25. The phantom according to claim 23, wherein the one or more simulated anatomical structures further comprise at least one simulated lung having high acoustic impedance.

26. The phantom according to claim 23, further comprising a simulated bone within the first material, the simulated bone having a greater hardness than the tissue mimicking soft material.

27. A method for using the phantom according to claim 23, the method comprising at least partially filling the simulated gastrointestinal tract with a known volume via at least one of the two ports and imaging the phantom while the simulated gastrointestinal tract is partially filled with the known volume.
